(19) United States
(12) Patent Application Publication
(10) Pub. No.: US 2024/0212852 A1
(43) Pub. Date: Jun. 27, 2024

(54) SYSTEMS AND METHODS FOR AUTOMATED SPINE SEGMENTATION AND ASSESSMENT OF DEGENERATION USING DEEP LEARNING

(71) Applicant: Subtle Medical, Inc., Menlo Park, CA (US)

(72) Inventors: Lei XIANG, Shanghai (CN); Kai WANG, Menlo Park, CA (US); Enhao GONG, Sunnyvale, CA (US)

(21) Appl. No.: 18/602,373

(22) Filed: Mar. 12, 2024

Related U.S. Application Data
(63) Continuation of application No. PCT/US2022/044599, filed on Sep. 23, 2022.
(60) Provisional application No. 63/250,056, filed on Sep. 29, 2021.

(51) Int. Cl. G16H 50/20; G06T 7/00; G06T 7/11; G06T 7/70

(52) U.S. Cl. CPC G16H 50/20; G06T 7/0012; G06T 7/11; G06T 7/70; G06T 2207/10088; G06T 2207/20084; G06T 2207/30012

(57) ABSTRACT

A deep learning-based system is provided for spine segmentation and classification. The method comprises: (a) receiving a medical image of a subject, where the medical image captures one or more structures of the subject; (b) applying a first deep network to the medical image and outputting a detection result, where the detection result comprises at least a segmentation map of the one or more structures and a location predicted for the one or more structures; (c) generating an input to a second deep network based at least in part on the location predicted in (b); and (d) predicting a degenerative condition for the one or more structures by processing the input using the second deep network.

### CROSS-REFERENCE TO RELATED APPLICATION

This application is a Continuation application of International Application No. PCT/US2022/044599 filed on Sep. 23, 2022, which claims priority to U.S. Provisional Application No. 63/250,056 filed on Sep. 29, 2021, the content of which is incorporated herein in its entirety.

### BACKGROUND

Spinal degenerative diseases, such as lumbar disc herniation (LDH) and other symptoms are increasingly affecting youth, as well as plaguing the elderly and sedentary office workers. Early intervention and management can effectively prevent or slow down the progression of the diseases. As a non-invasive examination method, magnetic resonance imaging (MRI) has the advantages of excellent soft tissue contrast, non-ionizing radiation, and high specificity and sensitivity to musculoskeletal diseases. It is suitable for routine screening of the general population and a reliable imaging method to prevent spinal degenerative diseases.

Artificial intelligence algorithms can help improve the consistency and qualification of the diagnosis during the clinical evaluation, and additionally have potential value for quantitative evaluation of intervention on spinal degenerative diseases.

### SUMMARY

The present disclosure provides methods and systems for detecting and classifying degenerative changes in the spine using artificial intelligence (AI) on magnetic resonance (MR) images. In particular, the targets classified or segmented by the method and system may include several kinds of targets, including, but not limited to normal vertebra1 bodies, degenerative vertebra1 bodies, normal intervertebral discs, protrusion of intervertebral discs and the like. The present disclosure provides an AI algorithm to predict the locations and classifications of the spine from MRI data with improved accuracy and performance.

In an aspect, a deep learning-based system is provided for spine segmentation and classification. The computer-implemented method comprises: (a) receiving a medical image of a subject, where the medical image captures one or more structures of the subject: (b) applying a first deep network to the medical image and outputting a detection result, where the detection result comprises at least a segmentation map of the one or more structures and a location predicted for the one or more structures: (c) generating an input to a second deep network based at least in part on the location predicted in (b): and (d) predicting a degenerative condition for the one or more structures by processing the input using the second deep network.

Another aspect of the present disclosure provides a non-transitory computer readable medium comprising machine executable code that, upon execution by one or more computer processors, implements any of the methods above or elsewhere herein. For example, the one or more processors may perform operations that comprise: (a) receiving a medical image of a subject, where the medical image captures one or more structures of the subject: (b) applying a first deep network to the medical image and outputting a detection result, where the detection result comprises at least a segmentation map of the one or more structures and a location predicted for the one or more structures: (c) generating an input to a second deep network based at least in part on the location predicted in (b): and (d) predicting a degenerative condition for the one or more structures by processing the input using the second deep network.

In some embodiments, the medical image includes a magnetic resonance image. In some embodiments, the one or more structures are spine structures. In some embodiments, the first deep network comprises a segmentation model with a dual regulation module. In some cases, the dual regulation module is trained to predict the location of the one or more spine structures. In some cases, the segmentation model is trained to predict the segmentation map of the one or more spine structures.

In some embodiments, the input to the second deep network comprises one or more patches generated from the medical image based at least in part on the location predicted in (b). In some embodiments, the input to the second deep network comprises one or more attention maps generated from the segmentation map. In some embodiments, the input to the second deep network comprises at least a second medical image of a view that is different from the first medical image. In some embodiments, the second deep network comprises a plurality of branches. In some cases, the input comprises patches of at least two different sizes and wherein at least two of the plurality of branches are configured to process the patches of at least two different sizes respectively. In some cases, the input comprises patches of at least two different views and wherein at least two of the plurality of branches are configured to process the patches of at least two different views respectively.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a framework for detecting and classifying degenerative conditions (e.g., degenerative discs and vertebrae) in spine MR images.

FIG. 2 shows an exemplary pipeline of the segmentation network-based structure detection framework.

FIG. 3 shows exemplary model architecture of a detection framework.

FIG. 4 shows an example of a classification framework.

FIG. 5 shows an example of vertebrae and disc detection results.

FIG. 6 shows classification result comparison between different models.

FIG. 7 schematically illustrates an example MR system and a platform for implementing the methods consistent with those described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides methods and systems for detecting and classifying degenerative changes in a body part such as spine using artificial intelligence on MR images. In some cases, the methods and systems herein may be capable of detecting and classifying several kinds of targets, including but not limited to, normal vertebra1 bodies, degenerative vertebra1 bodies, normal intervertebral discs, protrusion of intervertebral discs and the like. In some embodiments, an AI algorithm of the present disclosure may be developed to predict the locations and classifications of the spine from MRI data.

The methods and systems herein may provide advantages over existing methods. First, the methods and systems may provide better detection and classification capability and may provide an automatic spine diagnosis report without the need for human intervention. Second, the detection component with a dual regression regulation module herein beneficially detects the location of targets such as vertebra and disc by eliminating the false positive prediction, thereby improving the detection accuracy. Third, the multi-view multi-scale feature and attention map-based degeneration classification framework herein may allow the system to facilitate the network training to better extract useful and meaningful features, thus resulting in superior spine classification results.

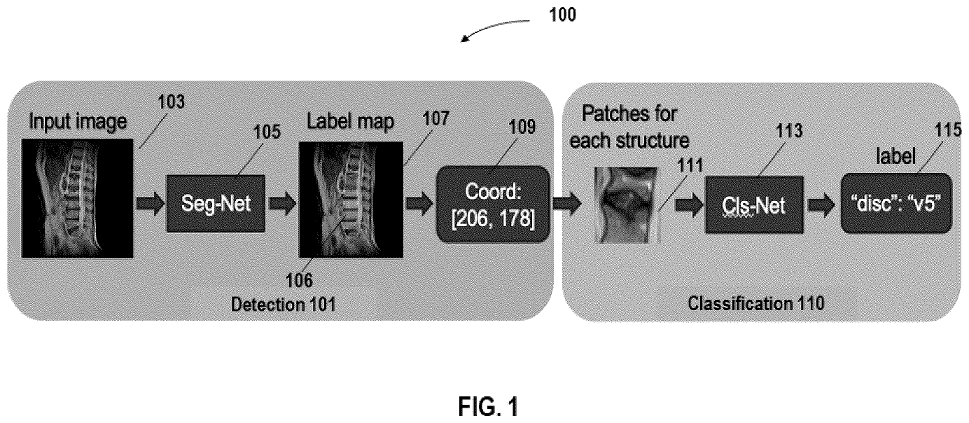

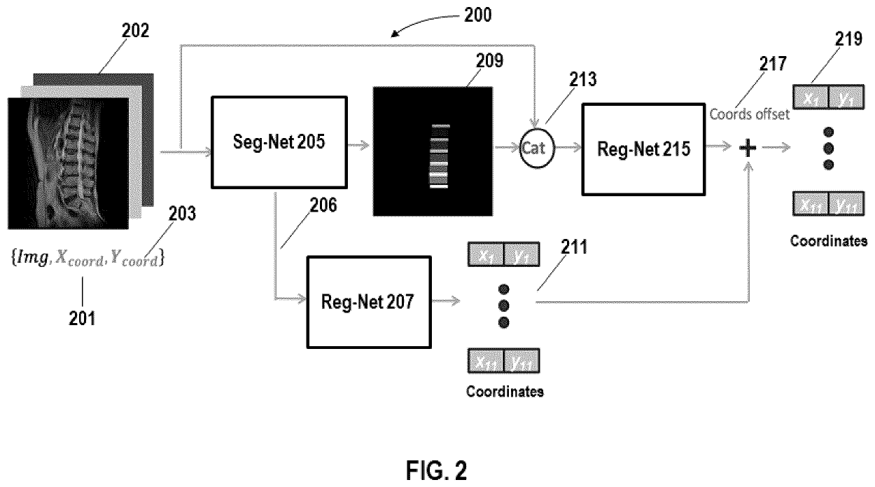

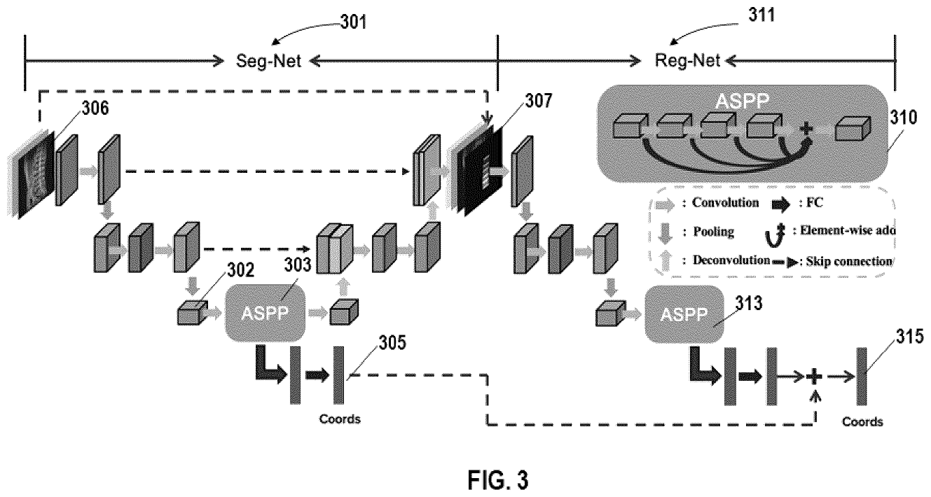

FIG. 1 shows an example of a framework 100 for detecting and classifying degenerative discs and vertebrae in spine MR images. The framework 100 may comprise a detection component 101 and a classification component 110. In some cases, the detection component 101 may comprise a segmentation network 105 trained to detect spine structures. The segmentation network 105 may, for example, take MR image (e.g., sagittal T1 image) as input 103 and generate an output. The output may comprise a segmentation map 107 and predicted locations (e.g., x, y coordinates) of the identified spine structures 109. In the illustrated example, the segmentation map may comprise labeled structure. For instance, the spine structures segmented by the network may be labeled. The segmented structures may be, for example, overlaid with visual indicators such as color-coded visual indicators 106 to make the segmented structures easily discernable. It should be noted various other suitable visual indicators (e.g., in any shape, color, text or image) may be utilized to indicate the segmented structures. In some embodiments, the segmentation-network 105 may comprise a dual regression regulation module which helps to improve the detection result. Details about segmentation-network are described with respect to FIG. 2 and FIG. 3.

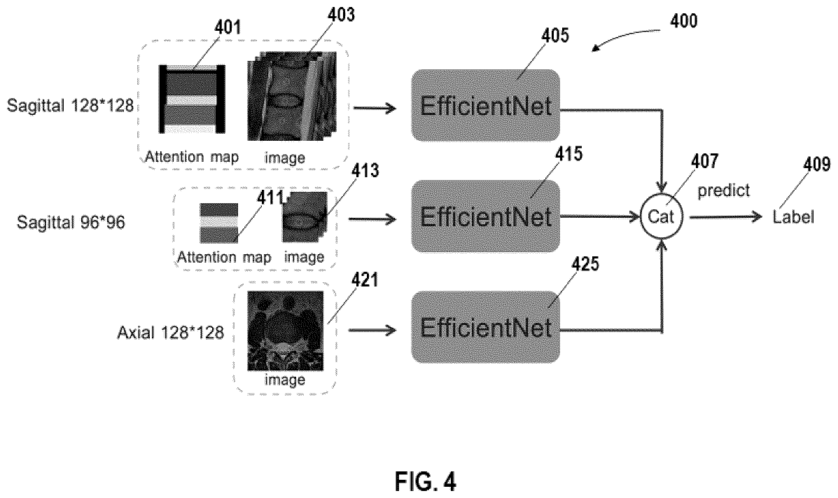

The framework 100 may also comprise a classification component 110 for classification tasks. The classification tasks may include predicting degenerative changes or degenerative conditions. The input to the classification component 110 may comprise one or more patches for each structure detected by the detection component 101. The one or more patches may be extracted based at least in part on the locations (coordinates) 109 of the structures predicted by the seg-net 105. In some cases, the segmentation map 107 may also be utilized by the classification network 113. For example, the segmentation maps 107 may be utilized as attention maps for the classification network 113 to highlight the useful features in the extracted patches. The output of the classification network may be the degenerative condition. For example, the output may comprise degenerative label of each structure 115. Details about the classification network 113 are described with respect to FIG. 4.

Though MR image, spine detection and degenerative changes classification examples are primarily provided herein, it should be understood that the present approach, models, methods and systems may be used in other imaging modality contexts. For instance, the presently described approach may be employed on data acquired by other types of tomographic scanners including, but not limited to, positron emission tomography (PET), computed tomography (CT), single photon emission computed tomography (SPECT) scanners, functional magnetic resonance imaging (fMRI) scanners and the like. Methods, systems and/or components of the systems or models may be used in other segmentation and classification tasks (e.g., disease condition prediction for other tissues, organs, etc.).

### Segmentation Network for Spine Structure Detection

FIG. 2 shows an exemplary pipeline of the segmentation network-based structure detection framework 200. The detection framework 200 may comprise a segmentation network 205 with dual regression regulation module (regularization networks 207, 215). In some cases, the input 201 to the segmentation network 205 may include MR images 202. For example, the input may include sagittal T1-weighted image. Depending on the tissues or structures to be imaged, other MR imaging sequences (e.g., T1-weighted. T2-weighted. FLAIR, etc.), view (e.g., axial, coronal, sagittal, etc.) or images acquired using other imaging modality may be the input. In some cases, the input 201 may also comprise x and y coordinate maps 203 and the input may be a multi-channel input. For example, the multiple channels may correspond to the x-y coordinate map and the sagittal T1-weighted image.

The segmentation network 205 may provide multiple outputs. In some cases, the outputs may comprise a structure segmentation map 209, and a high-level feature set (feature map) that encodes the location information of each structure 206. For instance, the structure segmentation map 209 may include spine structures detected by the segmentation network. For example, the structure segmentation map 209 may include 6 discs (i.e., T12-L1. L1-L2, L2-L3. L3-L4. L4-L5, and L5-S1) and 5 vertebrates (i.e., L1. L2, L3. L4, and L5). The high-level feature set 206 may be the input to a first regularization network 207. The first regularization network 207 may output a first coordinates prediction of the spine structures (e.g., 11 spine structures) 211. The output of the first regularization network 211 may comprise the location for each spine structure. For example, coordinates (e.g., x, y coordinate) for a middle point of the spine structure may be used as the location of the spine structure. It should be noted other point of a structure may be selected to represent the location of the structure. In some cases, the coordinate may be x, y coordinates. In some cases, the coordinate may be x, y, z coordinates.

In some embodiments, the input to the second regularization network 215 may comprise the generated segmentation map 209 along with the original input image 202. For example, the segmentation map 209 may be concatenated 213 with the original input image (e.g., sagittal T1 image 202) as the input to the second regularization network 215. The second regularization network 215 may predict the coordinate offset 217 or any difference between the first coordinate prediction 211 and the ground-truth coordinates. The second predication is the final structure location prediction 219. The structure location information may be used to extract patches for structure classification in the following steps.

FIG. 3 shows exemplary model architecture of the detection framework. The segmentation network 301 can be the same segmentation network as described in FIG. 2. The segmentation network 301 may be a U-Net network with the last layer of the U-Net network coding in traditional computing is replaced with ASPP (Atrous Spatial Pyramid Pooling). ASPP can effectively enlarge the receptive field without increasing the number of parameters. An example network structure of the ASPP 310 is illustrated in FIG. 3. The ASPP is used to perform convolution operation on the feature map of upper layer and may comprise five convolution processes. The first convolution may perform convolution calculation on the feature map (e.g., use 256 ordinary 1×1 convolution kernels), and add batch normalization layer operation after convolution. The second to fourth convolution calculations may use depthwise separable convolution calculations. In the fifth convolution process, the size of original image may be reduced to 1/output step size of previous size. The ASPP 310 may be the first regularization network that receives the high-level feature set (feature map) 302 that encodes the location information of each structure as input and output a first prediction of the coordinates 305 for each structure. The segmentation network 301 may predict a segmentation map 307. The segmentation map 307 may be concatenated with the original input image (e.g., sagittal T1 image 306) as the input to the second regularization network 311. The second regularization network may comprise ASPP 313 to predict the coordinate offset between the first coordinate prediction 305 and the ground-truth coordinates. The output 315 includes the coordinates for each detected structure (e.g., spine structures).

In some embodiments, a combination loss function of dice loss (L_{dice}) and multi-label cross entropy loss (L_{CE}) may be utilized to train the segmentation network. L₁ loss is utilized to train the regularization network. The total loss for training the whole network in the detection step may be formulated as follows:

$${L}_{DICE} ( ρ , \hat{ρ} ) = 1 - {\sum }_{c = 1}^{M} \frac{2 〈 {ρ}_{c} , {\hat{ρ}}_{c} 〉}{{‖ {ρ}_{c} ‖}_{2}^{2} + {‖ {\hat{ρ}}_{c} ‖}_{2}^{2}} \qquad ( 1 )$$

$${L}_{C E} ( ρ , \hat{ρ} ) = - {\sum }_{c = 1}^{M} {ρ}_{c} log log ( {\hat{ρ}}_{c} ) \qquad ( 2 )$$

$${L}_{d e t e c t i o n} = {L}_{C E} + α {L}_{DICE} + β {L}_{1} \qquad ( 3 )$$

where p is the manually labeled structure segmentation map (i.e., ground-truth) and \hat{p} is the predicted label map (e.g., segmentation map) by the proposed method. M is the number of classes. M can be any suitable number such as 5, 6, 7, 8, etc. α and β are the weights that balance L_{CE}-L_{DICE} and L₁. The weights may be determined such as based on experimental results. For instance, the weights may be adjusted based on the results on the validation dataset.

### Classification Network for Predicting Degenerative Changes

FIG. 4 shows an example of classification framework 400. The classification framework can the same as the classification component as described in FIG. 1. In some embodiments, the classification framework 400 may comprise multi-view multi-scale features and attention map. The input to the classification network may comprise image patches. In some cases, the image patches may be extracted for the detected structure (e.g., spine structure) according to the predicted structure coordinates from the detection step. In some cases, patches may be cropped image from the original image. For instance, a plurality of patches 403, 413, 421 may cropped from the original image according to a plurality of detected spine structures. In some embodiments, the input patches 403, 413 may have different scales. For instance, patches of different sizes may be created. In the illustrated example, two different patch size (e.g., 128*128 and 96*96) may be used to generate multi-scale features. In some cases, image patches of the two different sizes 403, 413 may be processed by two branches of the network. The patch size may be determined based at least in part on the image resolution (e.g., pixel size). For example, an image patch 413 with size of 96*96 may contain a spine structure, while an image patch 403 with size of 128*128 may contain the surrounding information of spine structure that useful for diagnosis. In some cases, an image patch may contain a single spine structure such as patch 413. Alternatively, an image patch may contain two or more spine structures such as patch 403.

In some embodiments, the classification network may utilize attention map 401, 411 that is generated based on the segmentation maps from the detection network. For example, a patch of image may be cropped from the segmentation map corresponding to the respective image patch 403, 413 and utilized as attention map 401, 411. In some cases, the attention map patches may be cropped also based on the predicted location of the structures. In some cases, the cropped segmentation map 401, 411 and the respective image patch 403, 413 may have the same size and correspond to the same detected structure(s). In some cases, the attention maps 401, 411 may be concatenated with the image patches 403, 413 to form part of the input data for the classification network. The method herein may utilize segmentation maps as attention maps to highlight the useful features in the extracted patches.

The classification framework 400 may comprise a plurality of branches. In some embodiments, the plurality of branches may comprise two or more branches to process the input image of different scales (e.g., different patch sizes). In some embodiments, the plurality of branches may be configured to process input images of different views (e.g., sagittal, axial, coronal, etc.). In some cases, at least two of the plurality of branches are configured to process image patches of different sizes but of the same view (e.g., sagittal patch 403, 413). In some cases, at least two of the plurality of branches are configured to process image patches of a first view (e.g., sagittal patch 403, 413) and a second view that is different from the first view (e.g., axial patch 421). The image patch of the different view may or may not have the same patch size of the other view. For instance, in order to utilize the meaningful feature from multi-view, the method may add image patch of a different view (e.g., axial patches 431) as the third branch of the classification network as shown in FIG. 3. In some cases, the axial patches may be extracted according to the locations predicted by the regularization network in the detection step.

In some embodiments, each branch of the classification framework may comprise an EfficientNet 405, 415, 425. The EfficientNets of the different branches may be different (e.g., three different EfficientNets 405, 415, 425). The high-level features generated by the different EfficientNets (e.g., three different EfficientNets) may be concatenated 407 together to predict the degenerative label 409 of each structure. An example of the EfficientNets architecture may comprise a main building block which is mobile inverted bottleneck MBConv with squeeze-and-excitation optimization. The different EfficinetNets 405, 415, 425 may be obtained by scaling a baseline model (e.g., scales all dimensions of depth/width/resolution using an effective compound coefficient) to different scaling dimensions based at least in part on the size of the input patches. For instance, for higher resolution images, network depth may be increased, such that the larger receptive fields can help capture similar features that include more pixels in bigger images. In some cases, the baseline network may be developed by leveraging a multi-objective neural architecture search that optimizes both accuracy and FLOPS.

### EXAMPLE

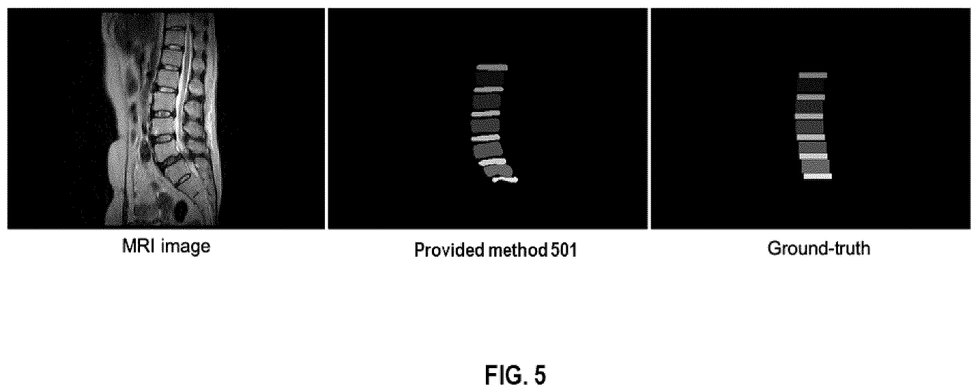

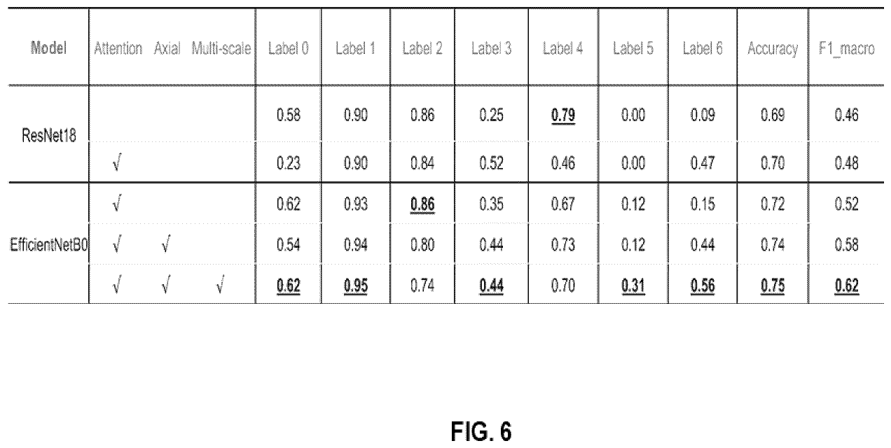

200 subjects were recruited from which 150 subjects were used for training and 50 subjects for testing. Vertebrae and disc position is marked on the MR image of T2 sagittal plane. Mark starts from disc of thoracic vertebral2(T12) lumbar vertebra1 (L1) to disc of lumbar vertebra5(L5) sacral vertebra1(S1). Vertebrae and discs are separated, such as L1, L2, etc. There are two kinds of vertebrae including normal vertebrae and degenerative vertebrae and five types of discs including normal disc, bulged disc, protruded disc, extruded disc and schmor. The detection results of vertebrae and discs are shown in Table. 1. FIG. 5 shows an example of vertebrae and disc detection results. As shown in the example, the method herein 501 with a dual regression regulation module integrated in the pipeline achieves improved detection result. FIG. 6 shows the classification result comparison between different models. As shown in the result, the method herein (EfficienNetB0) with attention map, axial view and multi-scale) achieves the best performance.

TABLE 1
Detection results of vertebrae and disc

| | Baseline 1 | Baseline 2 | + | | |
|---|---|---|---|---|---|
| Model | (Reg only) | (Seg only) | Morphological | + Reg | Proposed |
| Accuracy | 0.63 | 0.82 | 0.92 | 0.95 | 0.97 |

### System Overview

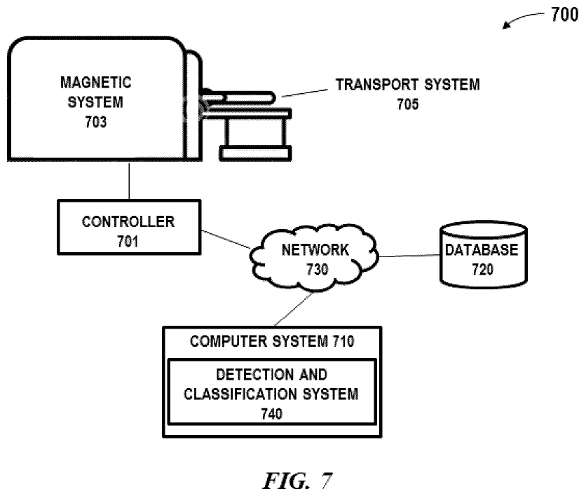

The systems and methods can be implemented on existing imaging systems such as but not limited to MR imaging systems without a need of a change of hardware infrastructure. Alternatively, the systems and methods can be implemented by any computing systems that may not be coupled to the MR imaging system. For instance, methods and systems herein may be implemented in a remote system, one or more computer servers, which can enable distributed computing, such as cloud computing. FIG. 7 schematically illustrates an example MR system 700 comprising a computer system 710 and one or more databases operably coupled to a controller over the network 730. The computer system 710 may be used for further implementing the methods and systems as described for processing the medical images (MR images) for detection and classification.

The controller 701 may be operated to provide the MRI sequence controller information about a pulse sequence and/or to manage the operations of the entire system, according to installed software programs. The controller may also serve as an element for instructing a patient to perform tasks, such as, for example, a breath hold by a voice message produced using an automatic voice synthesis technique. The controller may receive commands from an operator which indicate the scan sequence to be performed. The controller may comprise various components such as a pulse generator module which is configured to operate the system components to carry out the desired scan sequence, producing data that indicate the timing, strength and shape of the RF pulses to be produced, and the timing of and length of the data acquisition window. Pulse generator module may be coupled to a set of gradient amplifiers to control the timing and shape of the gradient pulses to be produced during the scan. Pulse generator module also receives patient data from a physiological acquisition controller that receives signals from sensors attached to the patient, such as ECG (electrocardiogram) signals from electrodes or respiratory signals from a bellows. Pulse generator module may be coupled to a scan room interface circuit which receives signals from various sensors associated with the condition of the patient and the magnet system. A patient positioning system may receive commands through the scan room interface circuit to move the patient to the desired position for the scan.

The controller 701 may comprise a transceiver module which is configured to produce pulses which are amplified by an RF amplifier and coupled to RF coil by a transmit/receive switch. The resulting signals radiated by the excited nuclei in the patient may be sensed by the same RF coil and coupled through transmit/receive switch to a preamplifier. The amplified nuclear magnetic resonance (NMR) signals are demodulated, filtered, and digitized in the receiver section of transceiver. Transmit/receive switch is controlled by a signal from pulse generator module to electrically couple RF amplifier to coil for the transmit mode and to preamplifier for the receive mode. Transmit/receive switch may also enable a separate RF coil (for example, a head coil or surface coil, not shown) to be used in either the transmit mode or receive mode.

The NMR signals picked up by RF coil may be digitized by the transceiver module and transferred to a memory module coupled to the controller. The receiver in the transceiver module may preserve the phase of the acquired NMR signals in addition to signal magnitude. The down converted NMR signal is applied to an analog-to-digital (A/D) converter (not shown) which samples and digitizes the analog NMR signal. The samples may be applied to a digital detector and signal processor which produces in-phase (I) values and quadrature (Q) values corresponding to the received NMR signal. The resulting stream of digitized I and Q values of the received NMR signal may then be employed to reconstruct an image. The provided methods herein may take the reconstructed image as input and process for detection and classification purpose.

The controller 701 may comprise or be coupled to an operator console (not shown) which can include input devices (e.g., keyboard) and control panel and a display. For example, the controller may have input/output (I/O) ports connected to an I/O device such as a display, keyboard and printer. In some cases, the operator console may communicate through the network with the computer system 710 that enables an operator to control the production and display of images on a screen of display.

The system 700 may comprise a user interface. The user interface may be configured to receive user input and output information to a user. The user input may be related to control of image acquisition. The user input may be related to the operation of the MRI system (e.g., certain threshold settings for controlling program execution, parameters for controlling the joint estimation of coil sensitivity and image reconstruction, etc). The user input may be related to various operations or settings about the detection and classification system 740. The user input may include, for example, a selection of a target structure or ROI, training parameters, displaying settings of a reconstructed image, customizable display preferences, selection of an acquisition scheme, and various others. The user interface may include a screen such as a touch screen and any other user interactive external device such as handheld controller, mouse, joystick, keyboard, trackball, touchpad, button, verbal commands, gesture-recognition, attitude sensor, thermal sensor, touch-capacitive sensors, foot switch, or any other device.

The MRI platform 700 may comprise computer systems 710 and database systems 720, which may interact with the controller. The computer system can comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, a plurality of processors for parallel processing, in the form of fine-grained spatial architectures such as a field programmable gate array (FPGA), an application-specific integrated circuit (ASIC), and/or one or more Advanced RISC Machine (ARM) processors. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit. 256 bit. 128 bit. 64 bit. 32 bit, or 16 bit data operations.

The imaging platform 700 may comprise one or more databases. The one or more databases 720 may utilize any suitable database techniques. For instance, structured query language (SQL) or “NoSQL” database may be utilized for storing image data, raw collected data, reconstructed image data, training datasets, validation dataset, trained model (e.g., hyper parameters), weighting coefficients, etc. Some of the databases may be implemented using various standard data-structures, such as an array, hash. (linked) list, struct, structured text file (e.g., XML), table. JSON. NOSQL and/or the like. Such data-structures may be stored in memory and/or in (structured) files. In another alternative, an object-oriented database may be used. Object databases can include a number of object collections that are grouped and/or linked together by common attributes: they may be related to other object collections by some common attributes. Object-oriented databases perform similarly to relational databases with the exception that objects are not just pieces of data but may have other types of functionality encapsulated within a given object. If the database of the present disclosure is implemented as a data-structure, the use of the database of the present disclosure may be integrated into another component such as the component of the present disclosure. Also, the database may be implemented as a mix of data structures, objects, and relational structures. Databases may be consolidated and/or distributed in variations through standard data processing techniques. Portions of databases. e.g., tables, may be exported and/or imported and thus decentralized and/or integrated.

The network 730 may establish connections among the components in the imaging platform and a connection of the imaging system to external systems. The network 730 may comprise any combination of local area and/or wide area networks using both wireless and/or wired communication systems. For example, the network 730 may include the Internet, as well as mobile telephone networks. In one embodiment, the network 730 uses standard communications technologies and/or protocols. Hence, the network 730 may include links using technologies such as Ethernet. 802.11, worldwide interoperability for microwave access (WiMAX). 2G/3G/4G mobile communications protocols, asynchronous transfer mode (ATM). InfiniBand. PCI Express Advanced Switching, etc. Other networking protocols used on the network 230 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS). Internet Protocol security (IPsec), etc. In another embodiment, the entities on the network can use custom and/or dedicated data communications technologies instead of, or in addition to, the ones described above.

Systems and methods of the present disclosure may provide a detection and classification system 740 that can be implemented in software, hardware, firmware, embedded hardware, standalone hardware, application specific-hardware, or any combination of these. The detection and classification system 740 can be a standalone system that is separate from the MR imaging system. The detection and classification system 740 may be in communication with the MR imaging system such as a component of a controller of the MR imaging system. In some embodiments, the detection and classification system 740 may comprise multiple components, including but not limited to, a training module, a detection and classification module and a user interface module.

The training module may be configured to train the detection and classification model framework as described above. For instance, the training module may be configured to train a detection network for spine structure detection and classification network for predicting a degenerative condition. The training module may train the two models or networks separately. Alternatively or in addition to, the two models may be trained as an integral model.

The training module may be configured to obtain and manage training datasets. For example, the training datasets for the detection network may comprise pairs of ground truth segmentation map and MR image from same subject. The training module may be configured to train the detection network and classification network as described elsewhere herein. The training module may train a model off-line. Alternatively or additionally, the training module may use real-time data as feedback to refine the model for improvement or continual training.

The detection and classification module may be configured to perform detection and classification (e.g., degenerative condition prediction) using trained models obtained from the training module. The detection and classification module may deploy and implement the trained model for making inferences. e.g., predicting locations for detected spine structures and prediction degenerative condition for each of the detected spine structure.

The user interface module may permit users to view the training result, view predicted results or interact with the training process.

Methods as described herein can be implemented by way of machine (e.g., computer processor) executable code stored on an electronic storage location of the computer system 710, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables: copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein A and/or B encompasses one or more of A or B, and combinations thereof such as A and B. It will be understood that although the terms “first,” “second,” “third” etc. are used herein to describe various elements, components, regions and/or sections, these elements, components, regions and/or sections should not be limited by these terms. These terms are merely used to distinguish one element, component, region or section from another element, component, region or section. Thus, a first element, component, region or section discussed herein could be termed a second element, component, region or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising.” or “includes” and/or “including.” when used in this specification, specify the presence of stated features, regions, integers, steps, operations, elements and/or components, but do not preclude the presence or addition of one or more other features, regions, integers, steps, operations, elements, components and/or groups thereof.

Reference throughout this specification to “some embodiments,” or “an embodiment.” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method for spine segmentation and classification, the method comprising:
(a) receiving a medical image of a subject, wherein the medical image captures one or more structures of the subject;
(b) applying a first deep network to the medical image and outputting a detection result, wherein the detection result comprises at least a segmentation map of the one or more structures and a location predicted for the one or more structures;
(c) generating an input to a second deep network based at least in part on the location predicted in (b); and
(d) predicting a degenerative condition for the one or more structures by processing the input using the second deep network.

2. The computer-implemented method of claim 1, wherein the medical image includes a magnetic resonance image and the one or more structures comprise one or more spine structures.

3. The computer-implemented method of claim 1, wherein the first deep network comprises a segmentation model with a dual regulation module.

4. The computer-implemented method of claim 3, wherein the dual regulation module is trained to predict the location of the one or more structures.

5. The computer-implemented method of claim 3, wherein the segmentation model is trained to predict the segmentation map of the one or more structures.

6. The computer-implemented method of claim 1, wherein the input to the second deep network comprises one or more patches generated from the medical image based at least in part on the location predicted in (b).

7. The computer-implemented method of claim 1, wherein the input to the second deep network comprises one or more attention maps generated from the segmentation map.

8. The computer-implemented method of claim 1, wherein the input to the second deep network comprises at least a second medical image of a view that is different from the first medical image.

9. The computer-implemented method of claim 1, wherein the second deep network comprises a plurality of branches.

10. The computer-implemented method of claim 9, wherein the input comprises patches of at least two different sizes and wherein at least two of the plurality of branches are configured to process the patches of at least two different sizes respectively.

11. The computer-implemented method of claim 9, wherein the input comprises patches of at least two different views and wherein at least two of the plurality of branches are configured to process the patches of at least two different views respectively.

12. A non-transitory computer-readable storage medium including instructions that, when executed by one or more processors, cause the one or more processors to perform operations comprising:
(a) receiving a medical image of a subject, wherein the medical image captures one or more structures of the subject;
(b) applying a first deep network to the medical image and outputting a detection result, wherein the detection result comprises at least a segmentation map of the one or more structures and a location predicted for the one or more structures;
(c) generating an input to a second deep network based at least in part on the location predicted in (b); and
(d) predicting a degenerative condition for the one or more structures by processing the input using the second deep network.

13. The non-transitory computer-readable storage medium of claim 12, wherein the medical image includes a magnetic resonance image and wherein the one or more structures are spine structures.

14. The non-transitory computer-readable storage medium of claim 12, wherein the first deep network comprises a segmentation model with a dual regulation module.

15. The non-transitory computer-readable storage medium of claim 14, wherein the dual regulation module is trained to predict the location of the one or more structures.

16. The non-transitory computer-readable storage medium of claim 14, wherein the segmentation model is trained to predict the segmentation map of the one or more structures.

17. The non-transitory computer-readable storage medium of claim 12, wherein the input to the second deep network comprises one or more patches generated from the medical image based at least in part on the location predicted in (b).

18. The non-transitory computer-readable storage medium of claim 12, wherein the input to the second deep network comprises one or more attention maps generated from the segmentation map.

19. The non-transitory computer-readable storage medium of claim 12, wherein the input to the second deep network comprises at least a second medical image of a view that is different from the first medical image.

20. The non-transitory computer-readable storage medium of claim 12, wherein the second deep network comprises a plurality of branches.
